(19) United States
(12) Patent Application Publication
(10) Pub. No.: US 2003/0017556 A1
(43) Pub. Date: Jan. 23, 2003

(54) Process for the preparation of L-amino acids using strains of the enterobacteiaceae family

(76) Inventor: Thomas Hermann, Bielefeld (DE)

Correspondence Address: SMITH, GAMBRELL & RUSSELL, LLP, 1850 M STREET, N.W., SUITE 800, WASHINGTON, DC, 20036, US

(21) Appl. No.: 10/186,999

(22) Filed: Jul. 3, 2002

(30) Foreign Application Priority Data
Jul. 6, 2001 (DE) 101 32 945.8

(51) Int. Cl. C12P 13/04; C12N 1/21

(52) U.S. Cl. 435/106; 435/252.3; 435/252.33

(57) ABSTRACT

The invention relates to a process for the fermentative preparation of L-amino acids, in particular L-threonine.

### BACKGROUND OF THE INVENTION

This invention relates to a process for the preparation of L-amino acids, in particular L-threonine, using strains of the Enterobacteriaceae family in which at least one or more of the genes chosen from the group consisting of dps, hns, lrp, pgm, fba, ptsG, ptsH, ptsI, crr, mopB, ahpC and ahpF is (are) attenuated. All references cited herein are expressly incorporated by reference. Incorporation by reference is also designated by the term “I.B.R.” following any citation.

L-Amino acids, in particular L-threonine, are used in human medicine and in the pharmaceuticals industry, in the foodstuffs industry and very particularly in animal nutrition.

It is known to prepare L-amino acids by fermentation of strains of Enterobacteriaceae, in particular Escherichia coli (E. coli) and Serratia marcescens. Because of their great importance, work is constantly being undertaken to improve the preparation processes. Improvements to the process can relate to fermentation measures, such as e.g. stirring and supply of oxygen, or the composition of the nutrient media, such as e.g. the sugar concentration during the fermentation, or the working up to the product form, by e.g. ion exchange chromatography, or the intrinsic output properties of the microorganism itself.

Methods of mutagenesis, selection and mutant selection are used to improve the output properties of these microorganisms. Strains which are resistant to antimetabolites, such as e.g. the threonine analogue α-amino-β-hydroxyvaleric acid (AHV), or are auxotrophic for metabolites of regulatory importance and produce L-amino acids, such as e.g. L-threonine, are obtained in this manner.

Methods of the recombinant DNA technique have also been employed for some years for improving the strain of strains of the Enterobacteriaceae family which produce L-amino acids, by amplifying individual amino acid biosynthesis genes and investigating the effect on the production.

The invention provides new measures for improved fermentative preparation of L-amino acids, in particular L-threonine.

### BRIEF SUMMARY OF THE INVENTION

The invention provides a process for the preparation of L-amino acids, in particular L-threonine, using microorganisms of the Enterobacteriaceae family which in particular already produce L-amino acids and in which at least one or more of the nucleotide sequence(s) which code(s) for the genes dps, hns, lrp, pgm, fba, ptsG, ptsH, ptsI, crr, mopB, ahpC and ahpF is (are) attenuated.

### DETAILED DESCRIPTION OF THE INVENTION

Where L-amino acids or amino acids are mentioned in the following, this means one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Threonine is particularly preferred.

The term “attenuation” in this connection describes the reduction or elimination of the intracellular activity of one or more enzymes (proteins) in a microorganism, such as bacteria, which are coded by the corresponding DNA, for example by using a weak promoter or a gene or allele which codes for a corresponding enzyme with a low activity or inactivates the corresponding enzyme (protein) or gene, and optionally combining these measures.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

The process is characterized in that the following steps are carried out:

a) fermentation of microorganisms of the Enterobacteriaceae family in which at least one or more of the genes chosen from the group consisting of dps, hns, lrp, pgm, fba, ptsG, ptsH, ptsI, crr, mopB, ahpC and ahpF is (are) attenuated,

b) concentration of the corresponding L-amino acid in the medium or in the cells of the microorganisms of the Enterobacteriaceae family, and

c) isolation of the desired L-amino acid, constituents of the fermentation broth and/or the biomass in its entirety or portions (>0 to 100%) thereof optionally remaining in the product.

The microorganisms (i.e. bacteria) which the present invention provides can produce L-amino acids from glucose, sucrose, lactose, fructose, maltose, molasses, optionally starch, optionally cellulose or from glycerol and ethanol. They are representatives of the Enterobacteriaceae family chosen from the genera Escherichia, Erwinia, Providencia and Serratia. The genera Escherichia and Serratia are preferred. Of the genus Escherichia the species Escherichia coli and of the genus Serratia the species Serratia marcescens are to be mentioned in particular.

Suitable strains, which produce L-threonine in particular, of the genus Escherichia, in particular of the species Escherichi coli, are, for example

Escherichi coli TF427

Escherichi coli H4578

Escherichi coli KY10935

Escherichi coli VNIIgenetika MG442

Escherichi coli VNIIgenetika M1

Escherichi coli VNIIgenetika 472T23

Escherichi coli BKIIM B-3996

Escherichi coli kat 13

Escherichi coli KCCM-10132.

Suitable L-threonine-producing strains of the genus Serratia, in particular of the species Serratia marcescens, are, for example

Serratia marcescens HNr21

Serratia marcescens TLr156

Serratia marcescens T2000.

Strains from the Enterobacteriaceae family which produce L-threonine preferably have, inter alia, one or more genetic or phenotypic features chosen from the group consisting of: resistance to α-amino-β-hydroxyvaleric acid, resistance to thialysine, resistance to ethionine, resistance to α-methylserine, resistance to diaminosuccinic acid, resistance to α-aminobutyric acid, resistance to borrelidin, resistance to rifampicin, resistance to valine analogues, such as, for example, valine hydroxamate, resistance to purine analogues, such as, for example, 6-dimethylaminopurine, a need for L-methionine, optionally a partial and compensable need for L-isoleucine, a need for meso-diaminopimelic acid, auxotrophy in respect of threonine-containing dipeptides, resistance to L-threonine, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity to fluoropyruvate, defective threonine dehydrogenase, optionally an ability for sucrose utilization, enhancement of the threonine operon, enhancement of homoserine dehydrogenase I-aspartate kinase I, preferably of the feed back resistant form, enhancement of homoserine kinase, enhancement of threonine synthase, enhancement of aspartate kinase, optionally of the feed back resistant form, enhancement of aspartate semialdehyde dehydrogenase, enhancement of phosphoenol pyruvate carboxylase, optionally of the feed back resistant form, enhancement of phosphoenol pyruvate synthase, enhancement of transhydrogenase, enhancement of the RhtB gene product, enhancement of the RhtC gene product, enhancement of the YfiK gene product, enhancement of a pyruvate carboxylase, and attenuation of acetic acid formation.

It has been found that microorganisms of the Enterobacteriaceae family produce L-amino acids, in particular L-threonine, in an improved manner after attenuation, in particular elimination, of at least one or more of the genes chosen from the group consisting of dps, hns, lrp, pgm, fba, ptsG, ptsH, ptsI, crr, mopB, ahpC and ahpF.

The use of endogenous genes is in general preferred. The term “endogenous genes” or “endogenous nucleotide sequences” is understood to mean the genes or nucleotide sequences present in the population of a species.

The nucleotide sequences of the genes of Escherichia coli belong to the prior art and can also be found in the genome sequence of Escherichi coli published by Blattner et al. (Science 277: 1453-1462 (1997)) I.B.R.

| | |
|---|---|
| dps gene: | |
| Description: | Global regulator, hunger coiditions, DNA |
| | binder protein |
| Reference: | Almiron et al.; Genes & Development 6 (12B) |
| | 2646-54 (1992) I. B. R. |
| Accession No.: | AE000183 |
| Alternative gene names: | pexB, vtm |
| hns gene: | |
| Description: | DNA-binding protein HLP-II (RU, BH2, HD, |
| | NS); pleiotropic regulator (histone-like |
| | protein) |
| Reference: | Pon et al.; Molecular and General Genetics |
| | 212 (2): 199-202 (1988) I. B. R. |
| Accession No.: | AE000222 |
| Alternative gene names: | bglY, cur, drc, drdX, drs, fimG, |
| | mysA, osmZ, pilG, topX, virR |
| lrp gene: | |
| Description: | Regulator for the leucine regulon and high- |
| | affinity transport systems of branched- |
| | chain amino acids (leucine-responsive |
| | regulatory protein) |
| Reference: | Willins et al.; Journal of Biological |
| | Chemistry 266 (17): 10768-74 (1991) I. B. R. |
| | Wang et al.; Journal of Bacteriology |
| | 176 (7): 1831-1839 (1994) I. B. R. Friedberg |
| | et al.; Journal of Bacteriology 177 (6): 1624- |
| | 1626 (1995) I. B. R. Calvo und Matthews; |
| | Microbiological Reviews 58 (3): 466-490 |
| | (1994) I. B. R. Azam et al.; Journal of |
| | Bacteriology 181 (20): 6361-6370 (1999) |
| | I. B. R. |
| Accession No.: | AE000191 |
| Alternative gene names: | ihb, livR, lss, lstR, oppI, rblA, |
| | mbf |
| pgm gene: | |
| Description: | Phosphoglucomutase |
| EC No.: | 5.4.2.2 |
| Reference: | Lu and Kieckner, Journal of Bacteriology |
| | 176: 5847-5851 (1994) I. B. R. Brautaset et |
| | al.; Biotechnology and Bioengineering 58 (2- |
| | 3): 299-302 (1998) I. B. R. |
| Accession No.: | AE000172 |
| fba gene: | |
| Description: | Fructose bisphosphate aldolase (class II) |
| EC No.: | 4.1.2.13 |
| Reference: | Alefounder et al., Biochemical Journal |
| | 257: 529-34 (1989) I. B. R. Zgiby et al.; |
| | European Journal of Biochemistry 267 (6): |
| | 1858-1868 (2000) I. B. R. Baldwin et al.; |
| | Biochemical Journal 169 (3): 633-641 (1978) |
| | I. B. R. |
| Accession No.: | AE000376 |
| Alternative gene names: | fda, ald |
| ptsG gene: | |
| Description: | PTS system, glucose-specific IIBC |
| | component |
| Reference: | Erni and Zanolari; Journal of Biological |
| | Chemistry 261 (35): 16398-16403 (1986) |
| | I. B. R. Bouma et al.; Proceedings of the |
| | National Academy of Sciences U.S.A. 84 (4) |
| | 930-934 (1987) I. B. R. Meins et al.; Journal |
| | of Biological Chemistry 263 (26): 12986- |
| | 12993 (1988) I. B. R. |
| Accession No.: | AE000210 |
| Alternative Gene names: | CR, car, cat, gpt, umg, glcA |
| ptsH gene: | |
| Description: | phosphohistidine protein hexose |
| | phosphotransferase, Phosphocarrier HPr |
| | protein of the phosphotransferase-Systems |
| | (PTS) |
| EC No.: | 2.7.1.69 |
| Reference: | Saffen et al.; Journal of Biological |
| | Chemistry 262 (33): 16241-53 (1987) I. B. R. |
| | Postma et al.; In: Neidhardt (ed), |
| | Escherichia coli and Salmonella, American |
| | Society for Microbiology, Washington, D.C., |
| | U.S.A.: 1149-1174 (1996) I. B. R. |
| Accession No.: | AE000329 |
| Alternative gene names: | ctr, hpr |
| ptsI gene: | |
| Description: | Phosphoenolpyruvat-Protein- |
| | Phosphotransferase, Enzym I of the |
| | Phosphotransferase-Systems (PTS) |
| EC No.: | 2.7.3.9 |
| Reference: | Saffen et al.; Journal of Biological |
| | Chemistry 262 (33): 16241-53 (1987) I. B. R. |
| | Postma et al.; In: Neidhardt (ed), |
| | Escherichia coli and Salmonella, American |
| | Society for Microbiology, Washington, D.C., |
| | U.S.A.: 1149-1174 (1996) |
| Accession No. : | AE000329 |
| Alternative gene names: | ctr |
| crr gene: | |
| Description: | glucose-specific IIA component (phospho- |
| | carrier protein for glucose) of the |
| | Phosphotransferase-Systems (PTS) |
| Reference: | Saffen et al.; Journal of Biological |
| | Chemistry 262 (33): 16241-53 (1987) I. B. R. |
| | Postma et al.; In: Neidhardt (ed), |
| | Escherichia coil and Salmonella, American |
| | Society for Microbiology, Washington, D.C., |
| | U.S.A.: 1149-1174 (1996) I. B. R. |
| Accession No. | AE000329 |
| Alternative gene names: | gsr, iex, tgs, treD |
| mopB gene: | |
| Description: | chaperone GroES, binds to heat-shock |
| | protein Hsp60 in the presence of Mg-ATP, |
| | suppresses ATPase activity |
| Reference: | Chandrasekhar et al.; Journal of Biological |
| | Chemistry 261 (26): 12414-9 (1986) I. B. R. |
| | LaRossa and Van Dyk; Molecular Micro- |
| | biology 5 (3): 529-534 (1991) I. B. R. |
| Accession No. : | AE000487 |
| Alternative gene names: | groE, groES, hdh, tabB |
| ahpC gene: | |
| Description: | C22-subunit of the alkyl hydroperoxide |
| | reductase, detoxification of hydroperoxides |
| EC No.: | 1.6.4.- |
| Reference: | Ferrante et al.; Proceedings of the |
| | National Academy of Sciences U.S.A. |
| | 92 (17): 7617-21 (1995) I. B. R. Poole und |
| | Ellis; Biochemistry 35 (1): 56-64 (1996) |
| | I. B. R. Nishiyama et al.; Journal of |
| | Bacteriology 183 (8): 2431-2438 (2001) |
| | I. B. R. |
| Accession No.: | AE000166 |
| ahpF gene: | |
| Description: | F52a-subunit of the alkyl hydroperoxide |
| | reductase; detoxification of hydroperoxides |
| Reference: | Ferrante et al.; Proceedings of the |
| | National Academy of Sciences U.S.A. |
| | 92 (17): 7617-21 (1995) I. B. R. Poole und |
| | Ellis; Biochemistry 35 (1): 56-64 (1996) |
| | I. B. R. Nishiyama et al.; Journal of |
| | Bacteriology 183 (8): 2431-2438 (2001) |
| | I. B. R. |
| Accession No.: | AE000166 |

The nucleic acid sequences can be found in the databanks of the National Center for Biotechnology Information (NCBI) of the National Library of Medicine (Bethesda, Md., USA), the nucleotide sequence databank of the European Molecular Biologies Laboratories (EMBL, Heidelberg, Germany or Cambridge, UK) or the DNA databank of Japan (DDBJ, Mishima, Japan).

The genes described in the text references mentioned can be used according to the invention. Alleles of the genes which result from the degeneracy of the genetic code or due to “sense mutations” of neutral function can furthermore be used.

To achieve an attenuation, for example, expression of the genes or the catalytic properties of the enzyme proteins can be reduced or eliminated. The two measures can optionally be combined.

The reduction in gene expression can take place by suitable culturing, by genetic modification (mutation) of the signal structures of gene expression or also by the antisense-RNA technique. Signal structures of gene expression are, for example, repressor genes, activator genes, operators, promoters, attenuators, ribosome binding sites, the start codon and terminators. The person skilled in the art can find information in this respect, inter alia, for example, in Jensen and Hammer (Biotechnology and Bioengineering 58: 191-195 (1998)) I.B.R., in Carrier and Keasling (Biotechnology Progress 15:58-64 (1999) I.B.R., Franch and Gerdes (Current Opinion in Microbiology 3:159-164 (2000)) I.B.R. and in known textbooks of genetics and molecular biology, such as, for example, the textbook of Knippers (“Molekulare Genetik [Molecular Genetics]”, 6th edition, Georg Thieme Verlag, Stuttgart, Germany, 1995) I.B.R. or that of Winnacker (“Gene und Klone [Genes and Clones]”, VCH Verlagsgesellschaft, Weinheim, Germany, 1990) I.B.R.

Mutations which lead to a change or reduction in the catalytic properties of enzyme proteins are known from the prior art. Examples which may be mentioned are the works of Qiu and Goodman (Journal of Biological Chemistry 272: 8611-8617 (1997)) I.B.R., Yano et al. (Proceedings of the National Academy of Sciences, USA 95: 5511-5515 (1998) I.B.R., Wente and Schachmann (Journal of Biological Chemistry 266: 20833-20839 (1991) I.B.R. Summarizing descriptions can be found in known textbooks of genetics and molecular biology, such as e.g. that by Hagemann (“Allgemeine Genetik [General Genetics]”, Gustav Fischer Verlag, Stuttgart, 1986) I.B.R.

Possible mutations are transitions, transversions, insertions and deletions. Depending on the effect of the amino acid exchange on the enzyme activity, “missense mutations” or “nonsense mutations” are referred to. Insertions or deletions of at least one base pair in a gene lead to “frame shift mutations”, which lead to incorrect amino acids being incorporated or translation being interrupted prematurely. If a stop codon is formed in the coding region as a consequence of the mutation, this also leads to a premature termination of the translation. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik [Molecular Genetics]”, 6th edition, Georg Thieme Verlag, Stuttgart, Germany, 1995) I.B.R., that by Winnacker (“Gene und Klone [Genes and Clones]”, VCH Verlagsgesellschaft, Weinheim, Germany, 1990) I.B.R. or that by Hagemann (“Allgemeine Genetik [General Genetics]”, Gustav Fischer Verlag, Stuttgart, 1986) I.B.R.

Suitable mutations in the genes, such as, for example, deletion mutations, can be incorporated into suitable strains by gene or allele replacement.

A conventional method is the method, described by Hamilton et al. (Journal of Bacteriology 171: 4617-4622 (1989)) I.B.R., of gene replacement with the aid of a conditionally replicating pSC101 derivative pMAK705. Other methods described in the prior art, such as, for example, those of Martinez-Morales et al. (Journal of Bacteriology 181: 7143-7148 (1999)) I.B.R. or those of Boyd et al. (Journal of Bacteriology 182: 842-847 (2000)) I.B.R., can likewise be used.

It is also possible to transfer mutations in the particular genes or mutations which affect expression of the particular genes into various strains by conjugation or transduction.

It may furthermore be advantageous for the production of L-amino acids, in particular L-threonine, with strains of the Enterobacteriaceae family to enhance one or more enzymes of the known threonine biosynthesis pathway or enzymes of anaplerotic metabolism or enzymes for the production of reduced nicotinamide adenine dinucleotide phosphate, in addition to the attenuation of one or more of the genes chosen from the group consisting of dps, hns, lrp, pgm, fba, ptsG, ptsH, ptsI, crr, mopB, ahpC and ahpF.

The term “enhancement” in this connection describes the increase in the intracellular activity of one or more enzymes or proteins in a microorganism which are coded by the corresponding DNA, for example by increasing the number of copies of the gene or genes, using a potent promoter or a gene which codes for a corresponding enzyme or protein with a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

Thus, for example, one or more of the genes chosen from the group consisting of

the thrABC operon which codes for aspartate kinase, homoserine dehydrogenase, homoserine kinase and threonine synthase (U.S. Pat. No. 4,278,765 I.B.R.),

the pyc gene which codes for pyruvate carboxylase (DE-A-19 831 609 I.B.R.),

the pps gene which codes for phosphoenol pyruvate synthase (Molecular and General Genetics 231:332 (1992) I.B.R.),

the ppc gene which codes for phosphoenol pyruvate carboxylase (Gene 31:279-283 (1984) I.B.R.),

the pntA and pntB genes which code for transhydrogenase (European Journal of Biochemistry 158:647-653 (1986) I.B.R.),

the rhtB gene which imparts homoserine resistance (EP-A-0 994 190 I.B.R.),

the mqo gene which codes for malate:quinone oxidoreductase (WO 02/06459 I.B.R.),

the rhtC gene which imparts threonine resistance (EP-A-1 013 765 I.B.R.),

the thrE gene of Corynebacterium glutamicum which codes for threonine export protein (WO 01/92545 I.B.R.), and

the gdh gene which codes for glutamate dehydrogenase (Nucleic Acids Research 11: 5257-5266 (1983) I.B.R.; Gene 23: 199-209 (1983) I.B.R.)

can be enhanced, in particular over-expressed.

It may furthermore be advantageous for the production of L-amino acids, in particular L-threonine, in addition to the attenuation of one or more of the genes chosen from the group consisting of dps, hns, lrp, pgm, fba, ptsG, ptsH, ptsI, crr, mopB, ahpC and ahpF, for one or more of the genes chosen from the group consisting of

the tdh gene which codes for threonine dehydrogenase (Ravnikar and Somerville, Journal of Bacteriology 169: 4716-4721 (1987) I.B.R.),

the mdh gene which codes for malate dehydrogenase (E.C. 1.1.1.37) (Archives in Microbiology 149: 36-42 (1987) I.B.R.),

the gene product of the open reading frame (orf) yjfA (Accession Number AAC77180 of the National Center for Biotechnology Information (NCBI, Bethesda, Md., USA)),

the gene product of the open reading frame (orf) ytfP (Accession Number AAC77179 of the National Center for Biotechnology Information (NCBI, Bethesda, Md., USA)),

the pckA gene which codes for the enzyme phosphoenol pyruvate carboxykinase (Journal of Bacteriology 172, 7151-7156 (1990) I.B.R.),

the poxB gene which codes for pyruvate oxidase (Nucleic Acids Research 14(13): 5449-5460 (1986) I.B.R.),

the aceA gene which codes for the enzyme isocitrate lyase (Journal of Bacteriology 170: 4528-4536 (1988) I.B.R.),

the dgsA gene which codes for the DgsA regulator of the phosphotransferase system (Bioscience, Biotechnology and Biochemistry 59: 256-251 (1995) I.B.R.), which is also known by the designation mlc gene,

the fruR gene which codes for the fructose repressor (Jahreis et al., Molecular and General Genetics 226, 332-336 (1991) I.B.R.), which is also known by the designation cra gene, and

the rpoS-Gen which codes for the Sigma³⁸-Factor (WO 01/05939 I.B.R.), also known as katF-Gen,

to be attenuated, in particular eliminated or for the expression thereof to be reduced.

It may furthermore be advantageous for the production of L-amino acids, in particular L-threonine, in addition to the attenuation of one or more of the genes chosen from the group consisting of dps, hns, lrp, pgm, fba, ptsG, ptsH, ptsI, crr, mopB, ahpC and ahpF, to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982 I.B.R.).

The microorganisms produced according to the invention can be cultured in the batch process (batch culture), the fed batch (feed process) or the repeated fed batch process (repetitive feed process). A summary of known culture methods is described in the textbook by Chmiel (Bioprozesstechnik 1. Einf\dot{u}hrung in die Bioverfahrenstechnik [Bioprocess Technology 1. Introduction to Bioprocess Technology (Gustav Fischer Verlag, Stuttgart, 1991) I.B.R.) or in the textbook by Storhas (Bioreaktoren und periphere Einrichtungen [Bioreactors and Peripheral Equipment] (Vieweg Verlag, Braunschweig/Wiesbaden, 1994) I.B.R.).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981) I.B.R.

Sugars and carbohydrates, such as e.g. glucose, sucrose, lactose, fructose, maltose, molasses, starch and optionally cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substance can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 25° C. to 45° C., and preferably 30° C. to 40° C. Culturing is continued until a maximum of L-amino acids or L-threonine has formed. This target is usually reached within 10 hours to 160 hours.

The analysis of L-amino acids can be carried out by anion exchange chromatography with subsequent ninhydrin derivation, as described by Spackman et al. (Analytical Chemistry, 30: 1190-1206 (1958)) I.B.R., or it can take place by reversed phase HPLC as described by Lindroth et al. (Analytical Chemistry 51: 1167-1174 (1979)) I.B.R.

The process according to the invention is used for the fermentative preparation of L-amino acids, such as, for example, L-threonine, L-isoleucine, L-valine, L-methionine, L-homoserine and L-lysine, in particular L-threonine.

This application claims priority to German Priority Document Application No. 101 32 945.8, filed on Jul. 6, 2001. This application also claims priority to U.S. Provisional Appln. No. 60/303,789 filed Jul. 10, 2001. The German Priority document and the U.S. Provisional document are both hereby incorporated by reference in their entirety.

What is claimed is:

1. A method for the fermentative preparation of an L-amino acid, comprising:
a) fermenting, in a medium, a microorganism of the Enterobacteriaceae family which produces the desired L-amino acid and in which one or more of the genes selected from the group consisting of dps, hns, lrp, pgm, fba, ptsG, ptsH, ptsI, crr, mopB, ahpC, and ahpF, or nucleotide sequences which code for one or more of said genes, is attenuated.

2. The method according to claim 1, further comprising
b) concentrating the desired L-amino acid in the medium or in the cells of the microorganism.

3. The method according to claim 2, further comprising
c) isolating the desired L-amino acid.

4. The method according to claim 1, wherein at least on gene is eliminated.

5. The method according to claim 1, wherein the microorganism include genes of the biosynthesis pathway of the desired L-amino acid that are enhanced.

6. The method according to claim 1, wherein the microorganism include genes of the metabolic pathways which reduce the formation of the desired L-amino acid that are at least partly eliminated.

7. The method according to claim 1, wherein the expression of a polynucleotide which codes for one or more of the genes selected from the group consisting of dps, hns, lrp, pgm, fba, ptsG, ptsH, ptsI, crr, mopB, ahpC and ahpF is attenuated.

8. The method according to claim 7, wherein expression of a polynucleotide which codes for one or more genes selected from dps, hns, lrp, pgm, fba, ptsG, ptsH, ptsI, crr, mopB, ahpC and ahpF is eliminated.

9. The method according to claim 1, wherein at least one of the regulatory and catalytic properties of the polypeptides for which the polynucleotides dps, hns, lrp, pgm, fba, ptsG, ptsH, ptsI, crr, mopB, ahpC and ahpF code is reduced.

10. The method according to claim 1, wherein the microorganism comprises, at the same time, one or more genes which are enhanced; wherein said at least one gene is selected from the group consisting of:
the thrABC operon which codes for aspartate kinase, homoserine dehydrogenase, homoserine kinase and threonine synthase,
the pyc gene which codes for pyruvate carboxylase,
the pps gene which codes for phosphoenol pyruvate synthase,
the ppc gene which codes-for phosphoenol pyruvate carboxylase,
the pntA and pntB genes which code for transhydrogenase,
the rhtB gene which imparts homoserine resistance,
the mqo gene which codes for malate:quinone oxidoreductase,
the rhtC gene which imparts threonine resistance,
the thrE gene which codes for threonine export protein, and
the gdhA gene which codes for glutamate dehydrogenase.

11. The method according to claim 9, wherein said at least one enhanced gene is overexpressed.

12. The method according to claim 1, wherein the microorganism comprises, at the same time, at least one gene which is attenuated; wherein said at least one or gene is selected from the group consisting of:
the tdh gene which codes for threonine dehydrogenase,
the mdh gene which codes for malate dehydrogenase,
the gene product of the open reading frame (orf) yjfA,
the gene product of the open reading frame (orf) ytfP,
the pckA gene which codes for phosphoenol pyruvate carboxykinase,
the poxB gene which codes for pyruvate oxidase,
the aceA gene which codes for isocitrate lyase,
the dgsA gene which codes for the DgsA regulator of the phosphotransferase system,
the fruR gene which codes for the fructose repressor, and
the rpoS-Gen which codes for the Sigma³⁸-Factor.

13. The method according to claim 9, wherein said at least one attenuated gene is eliminated or reduced in expression.

14. The method according to claim 1, wherein the L-amino acid is L-threonine.

15. The method according to claim 3, wherein at least one of constituents of the fermentation medium and a resulting biomass in its entirety or portions thereof is also isolated.

16. A method for the fermentative preparation of an L-amino acid, comprising:
a) fermenting, in a medium, a microorganism of the Enterobacteriaceae family which produces the desired L-amino acid and in which one or more of the genes selected from the group consisting of dps, hns, lrp, pgm, fba, ptsG, ptsH, ptsI, crr, mopB, ahpC, and ahpF, or nucleotide sequences which code for one or more of said genes, is attenuated;
b) concentrating the desired L-amino acid in the medium or in the cells of the microorganism; and
c) isolating the desired L-amino acid.
